(19) United States
(12) Patent Application Publication
(10) Pub. No.: US 2018/0289434 A1
(43) Pub. Date: Oct. 11, 2018

(54) SYSTEM AND METHOD FOR COORDINATING A SURGICAL PROCEDURE

(71) Applicants: Matti Palo, Covington, LA (US); Ron Brewer, Covington, LA (US)

(72) Inventors: Matti Palo, Covington, LA (US); Ron Brewer, Covington, LA (US)

(21) Appl. No.: 15/644,357

(22) Filed: Jul. 7, 2017

Related U.S. Application Data
(60) Provisional application No. 62/482,367, filed on Apr. 6, 2017.

(51) Int. Cl. A61B 34/00; G06F 19/00

(52) U.S. Cl. CPC A61B 34/25; G06Q 10/087; G06F 19/3481; G06F 19/322

(57) ABSTRACT

Embodiments described herein provide a surgical management and coordination system and method of implementation. The system includes a microprocessor and integrated memory which provide recommended surgical coordination templates generated in successive phases corresponding to the various phases of a surgical procedure based on a specific bone set, surgeon or medical center. The system further enables an industry set sheet having specific fields populated by the device manufacturer to coordinate an efficient assembly and implementation of actual and used inventory levels to efficiently manage through an automated platforms inventory levels and adjustments of specific instruments based on the defined medical procedure.

### CROSS-REFERENCE TO RELATED APPLICATIONS

This non-provisional application claims priority to provisional application No. 62/482,367 to Palo et. al., filed on Apr. 6, 2017, which is hereby incorporated by reference.

### FIELD

The present embodiments relate to a surgical management and coordination system, and in particular, to surgical management and coordination system comprised of a plurality of scalable surgical coordination templates which may customizable by the administratively controlled authorized medical personnel to assist in the efficient preparation and execution of surgical procedure.

### BACKGROUND

The scheduling and coordinating of a medical procedure is a multi-phased process which must be strictly adhered to ensure the proper inventory, equipment, and personnel who assist in the procedure and who are knowledgeable with its execution. Though thousands of medical procedures are conducted throughout the country every day, the processes followed to execute them are often not cohesive, cluttered, and manual in nature leading to inefficient time and cost management as well as an increase in risk to patient safety and health.

One of the factors that lead to this breakdown of communication between personnel is the multiple layers of correspondence that are often sent last minute using different platforms. Each of the personnel has specific requirements in the execution of a medical procedure, such as the scheduling of required personnel, capturing time charges, gathering appropriate instruments and materials, assisting in the execution of the medical procedure and capturing charges for billing.

Hospitals currently manage these processes through outdated systems such as a hand-written “preference cards” and “pick sheets.” The “pick sheet” is a method used to gather all the standard instruments and materials necessary for the case. A true “procedure card” describing the way an operating room is equipped and configured for a specific case and the process around the operating room does not exist. Sometimes there is a “preference card,” consisting usually of an index card with handwritten instructions written on the pick sheet. Although there is often an electronic version of the “pick sheet,” it is not easily modified which leads to infrequent updating and outdated information. Currently, there is no system that allows for the easy modification of the “preference card” or “pick sheet” for a surgical procedure.

Each surgical procedure requires a specific set of medical devices and instruments to aid in the various phases of surgery such as gauze, clamps, forceps, scissors, drapes, instruments, dressings, suture, and other miscellaneous supplies. While some procedures may have a common core set of instruments or supplies, most procedures require both specific instruments and quantities which must be inventoried, stocked, and prepared long before the procedure is performed. Further, the medical device and instruments may be altered based on a surgeon's preference or variations in available inventory at a medical center.

Most legacy systems used today are either a product of their individual IT department or licensed software from an electronic health record provider, which is designed to fit within an electronic medical record. Without continuous maintenance and updating of data, these systems fail to provide an accurate record of necessary instruments and materials required in the operating room for each procedure, accurate levels of inventory in the storerooms, and specification of surgeon preferences which not only result in increased time to perform surgeries but also compromises patient safety.

Today many of these surgical management systems are merely a compilation of cards and notes shared amongst the medical staff within a single medical center based on available inventory available at the location and acquired knowledge of surgeon preferences created from past user inputs. Without a more efficient system to coordinate and manage surgical procedures, the costly waste of materials, equipment and time will continue.

To accurately account for inventory and supplies required to be available in the operating room and those utilized for each medical procedure performed by each surgeon, an easily created and edited resource management database is necessary to account for each type of item and the applicable quantity required by the specific surgeon. As major medical centers may have hundreds of doctors with thousands of procedures being performed daily, the task of maintaining this information update is time-consuming on the inventory and medical staff. Further, the endless task of updating these procedures increase the time and effort of the medical staff who must sacrifice surgical procedure time and post-surgery time trying to locate and restock this inventory. In addition, there is the need to accurately and easily schedule cases and perform charge reconciliation.

### SUMMARY OF THE INVENTION

The embodiments provided disclose a system which enables a plurality of administratively controlled authorized users comprised of medical staff and representatives to schedule, prepare, coordinate and bill surgical procedures and inventory using a plurality of scalable surgical coordination templates. Each of the scalable coordination templates may be shared and edited by authorized users and allows for pre-populated data field based on past user inputs and surgeon preferences. The system includes a microprocessor including a memory module configured to store a plurality of pre-programmed medical and logistical data which coordinate and direct both the medical staff and medical centers in efficiently scheduling surgical cases, audit inventory, and ordering medical supplies.

The system further provides a detailed, instructional outline of procedures wherein hospitals, surgeons and surgical staff may sequentially transition a patient from the surgical clinician's office through the operating room to the recovery room while simultaneously generating a variety of sheets and cards with recommendations based on past inputs by hospital and surgical staff using an integrated collaboration filter. The variety of sheets and cards enables step-by-step tutorials, recommendations, or voice dictation to provide guidance between members of the surgical staff and a designated surgeon, as well as provide the patient with concise post-operative instructions for the accurate transmission of requisite information for recovery.

The system is further configured to customize the scalable surgical coordination templates instrument set sheets to a specific pre-defined hospital or surgeon using a recommender module with a collaborative filter; generate medical representative industry sheets to ensure the proper assembly and implementation of medical devices using a recommender module with a collaborative filter and a live interface while in the operating room; and generate an industry set sheet 29 by a medical manufacturer representative on the most effective manner to assemble and implement their medical device; and create customized operations cards to the specific pre-defined surgeon to capture and share all desired and recommended steps to perform a specific medical procedure; and perform charge reconciliation to account for surgical and anesthesia charges and provide efficient logistics when ordering/re-ordering surgical supplies and equipment. In addition, the system enables hospital and surgical staff to adjust inventory levels of items supplied to the operating room in preparation for a surgical procedure performed by a specific surgeon by adjusting the data entry field of one of the scalable surgical coordination templates, in which one or more of the quantities of the items used in the procedure differ from the pre-defined quantity of the item supplied for the medical procedure. When an inventory item necessary for a surgical procedure has been designated as “needed,” an action plan for procurement is generated on the pick sheet “need category.” The system is configured to enable the surgical staff to be ultimate decision maker.

Other aspects, advantages, and novel features of the embodiments will become apparent from the following detailed description in conjunction with the drawings.

### BRIEF DESCRIPTION OF THE DRAWINGS

A more complete understanding of the embodiments, and the attendant advantages and features thereof, will be more readily understood by reference to the following detailed description when considered in conjunction with the accompanying drawings wherein:

FIG. 1 is a schematic diagram of a system for coordinating a surgical procedure;

FIG. 2 is a flowchart depicting a method of creating a scalable surgical coordination templates;

FIG. 3 is a screen capture of a doctor operation card of the system;

FIG. 4 is a further embodiment of the doctor operation card of the system;

FIG. 5 is a further screen capture of a “doctor preparation sheet” of the system;

FIG. 5A is a screen capture of a pick sheet card of the system;

FIG. 5B is a further screen capture of a “pick sheet” used in the system further including manufacturing, cost, pull, and need categories;

FIG. 5C is a screen capture of “instrument set card” used in the system;

FIG. 6 is an alternative embodiment illustrating the plurality of data entry fields within the pick sheet;

FIG. 7 is an alternative embodiment illustrating the plurality of data entry fields within the procedure cards;

FIG. 8 is an alternative embodiment illustrating the plurality of data entry fields within the instrument set sheet;

FIG. 9 is an alternative embodiment illustrating the plurality of data entry fields within the instrument sheet;

FIG. 10 is an alternative embodiment illustrating a variety of functions available in the operation card template; and

FIG. 11. is an alternative embodiment illustrating a variety of functions available in the charge capture.

### DETAILED DESCRIPTION

The specific details of the single embodiment or variety of embodiments described herein are set forth in this application. Any specific details of the embodiments are used for demonstration purposes only, and no unnecessary limitation or inferences are to be understood therefrom. Furthermore, as used herein, relational terms, such as “first” and “second,” “top” and “bottom,” and the like, may be used solely to distinguish one entity or element from another entity or element without necessarily requiring or implying any physical or logical relationship, or order between such entities or elements.

The embodiments provide a system which enables medical staff to prepare and coordinate inventory and peri-operative surgical processes using a plurality of scalable surgical coordination templates which may be shared amongst authorized users during the various phases of a surgical procedure by automating the analysis, inventory, and recommended process using a plurality of surgical coordination templates. The system and scalable surgical coordination templates are designed to reduce and manage the man-hours required to continuously update surgeon preference cards, set inventory sheets, operations card or similar records tracking actual usage and pre-defined quantities by members of the surgical staff.

Further, the system reduces the number of clerical errors which occur from the time-consuming task of continuous template updates with the above-mentioned information by only making adjustment recommendations when actual usage of the inventory items differ by a predetermined range from the defined quantities. The system also creates efficiency by using a recommender module with a collaborative filter to recommend changes to the “pick sheet” and “preference card” for surgical instruments and materials regularly supplied in the operating room but unused, having to be restocked by members of surgical staff.

Traditional surgical management systems require operating room personnel to be present in the operating room to handle inventory levels, send the instruments to the sterilization department, and assist with inventorying all used/unused instruments. The current system 10 automates these time-consuming tasks and streamlines the process for creating both a “pick sheet” and “preference card” using an automated template system.

In addition, many procedures require a representative to be present in an operating room to manage inventory and implants specific to a case in which an implant might be utilized. The current system 10 automates these tasks formerly utilized by costly medical representatives by creating a system which streamlines the actual required inventory levels for a specific medical procedure which enable the medical staff to sort and restock inventory levels without the need for a medical sale represented to be present in the operating room.

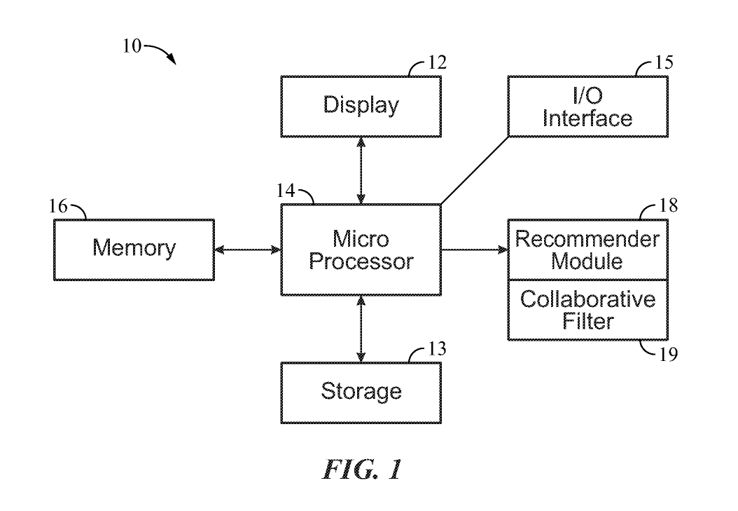

Referring now to the drawings wherein like reference numerals designate identical or corresponding parts throughout the views. There is shown in FIG. 1 a schematic diagram of the system for coordinating a surgical procedure 10 using a variety of doctor preparation cards which enable a user to populate selected fields viewable on a display 12 to effectively manage the medical procedure, location, surgeon, and required instruments. The system 10 and incorporated architecture control the various means for executing at least one function using a processor 14 to control the operation and management of the system 10. The processor 14 may include any general-purpose or special-purpose processor or microprocessor having an integrated memory module 16 configured to store a variety of executable code and data to the processor 14 relating to the management and coordination of a medical operation. The processor 14 may be connected to at least one input/out interface 15 which may further act to transmit and receive data, content, and images from the plurality of authorized users. Further, the inputted content from the plurality of authorized users may be collaborated over a wireless network and viewable on a number of wired or wireless devices allowing the user, surgical nurse, or representative to input a variety of medical data relating to procedure, instruments, surgeon preference or combination thereof. In the embodiment presented, it is contemplated the system will be distributed over a wired or wireless network such as a Wireless Local Area Network using an interface such as a computer, laptop or smart device which may be stored in a server/cloud-based server or computing device storage module.

The memory module 16 is configured to store a plurality of programmed information relating to the various medical procedures, surgeons, medical center, and pre-defined quantities of instruments available for a surgical procedure at a medical center. The system 10 enables the plurality of authorized users to receive a system administrative access and then select a medical center, surgeon, preferred medical representative concurrently with a plurality of generated surgical templates which recommend a variety of data entry inputs using a recommender module 18 and collaborative filter 19 in the medical center, and/or equipped by the medical device provider. It is contemplated the memory module 16 may include read-only memory (ROM), flash memory, or a variation of the random access memory (RAM) such as a static/dynamic or synchronous RAM. The memory module 16 can comprise volatile and/or non-volatile memory to store the plurality of successive phase forms including pick sheets, procedure cards, instrument set sheets, industry sheets, and operations cards. The memory module 16 may further be used to store surgeon preference cards and records including data recorded by a surgical nurse or representative who uses the items during the performance of the medical procedure. Further, the memory module 16 stores the application software to coordinate the activities, functions, task and all end-user programs associated with the medical procedure in accordance with the embodiments provided.

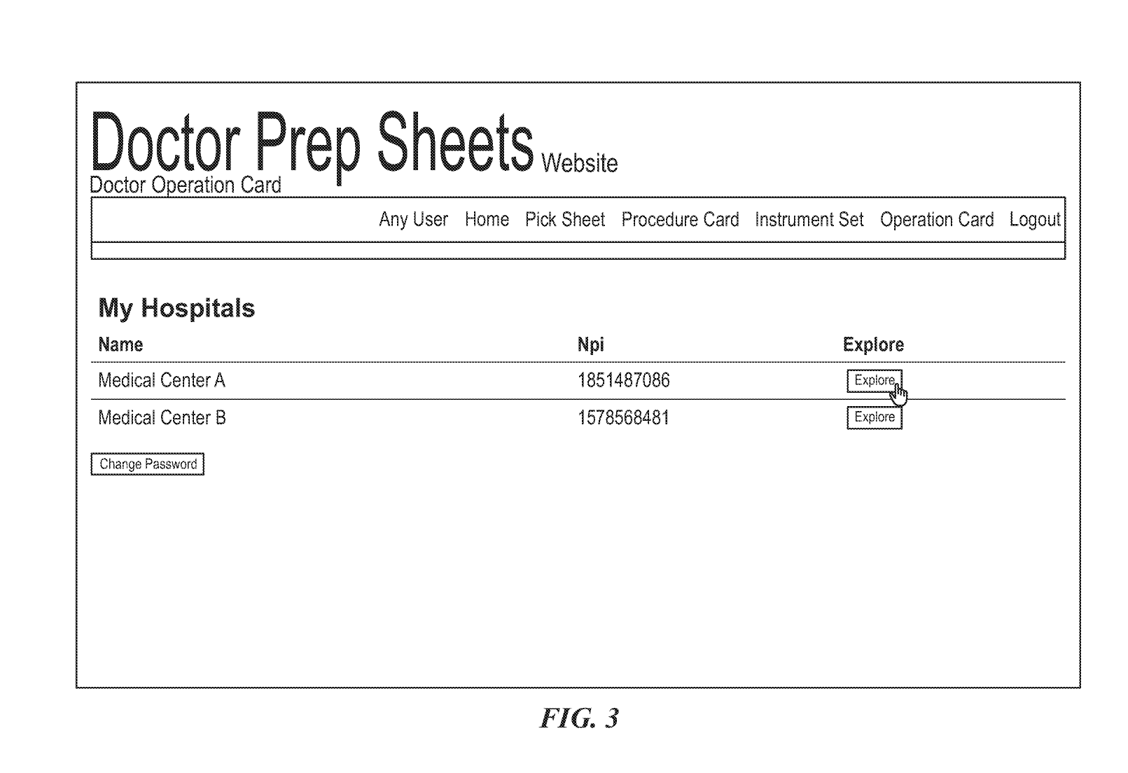

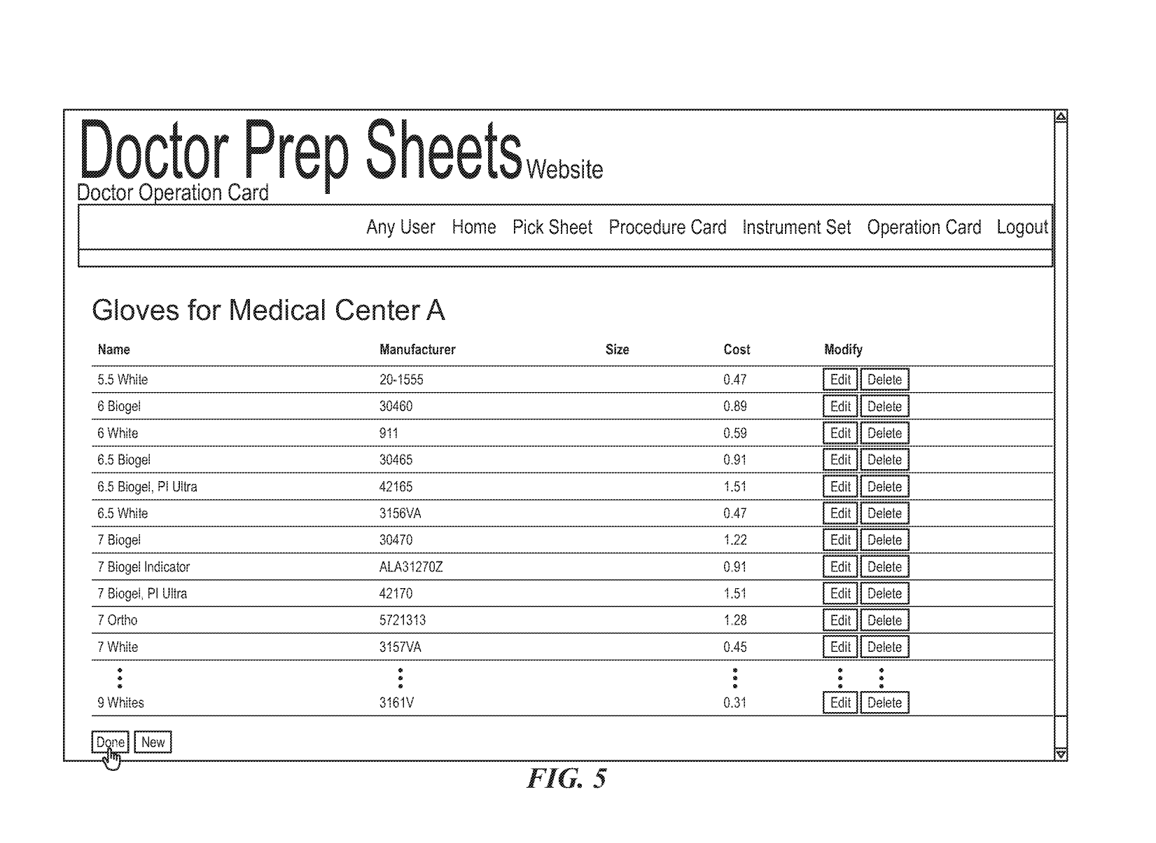

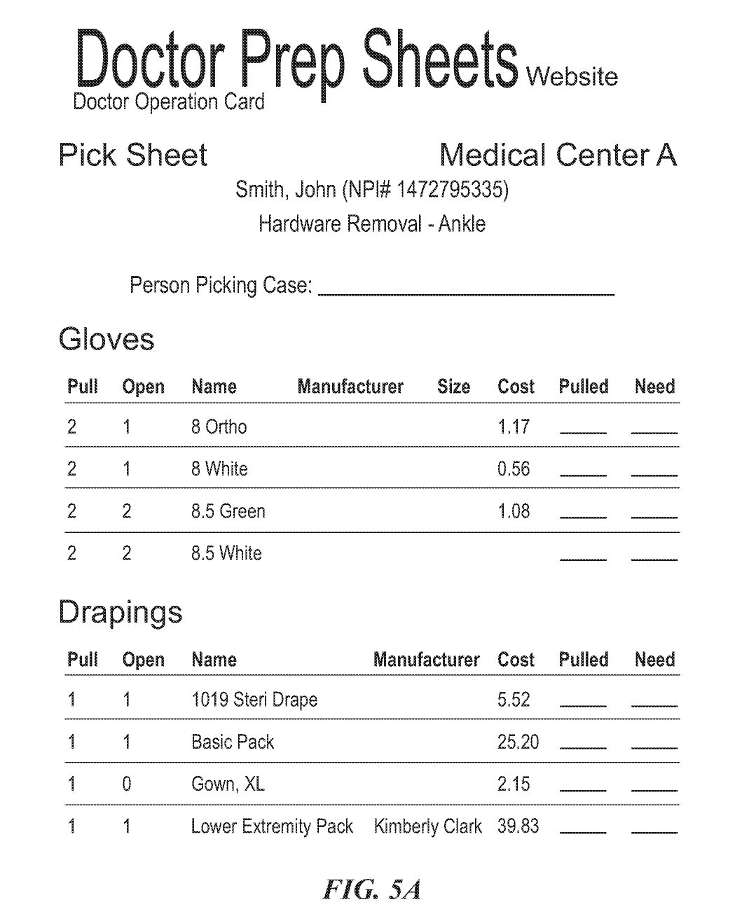

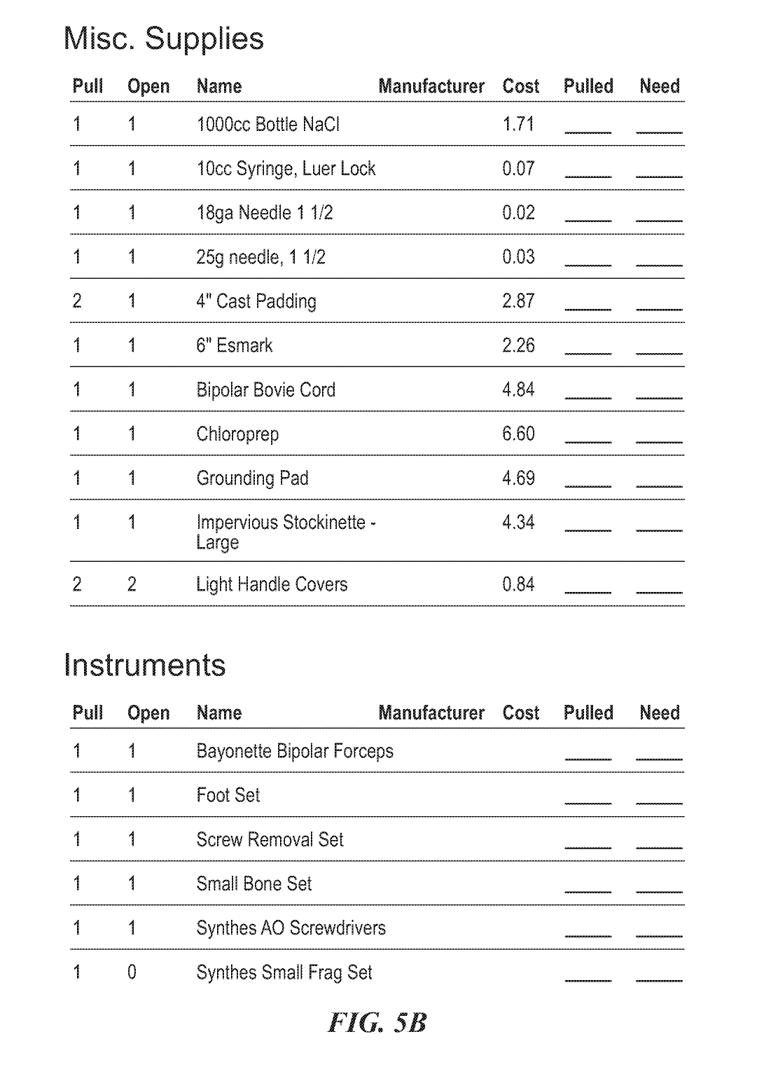

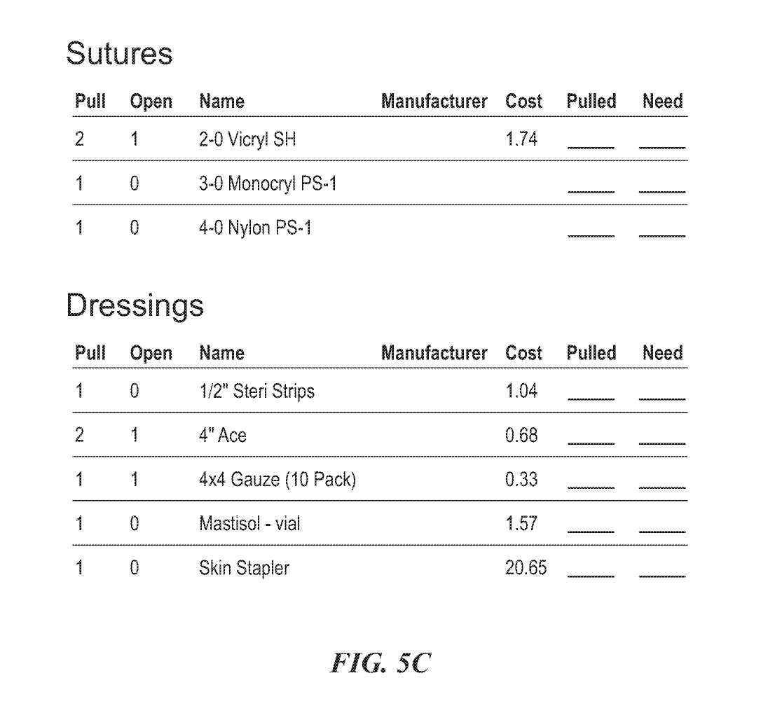

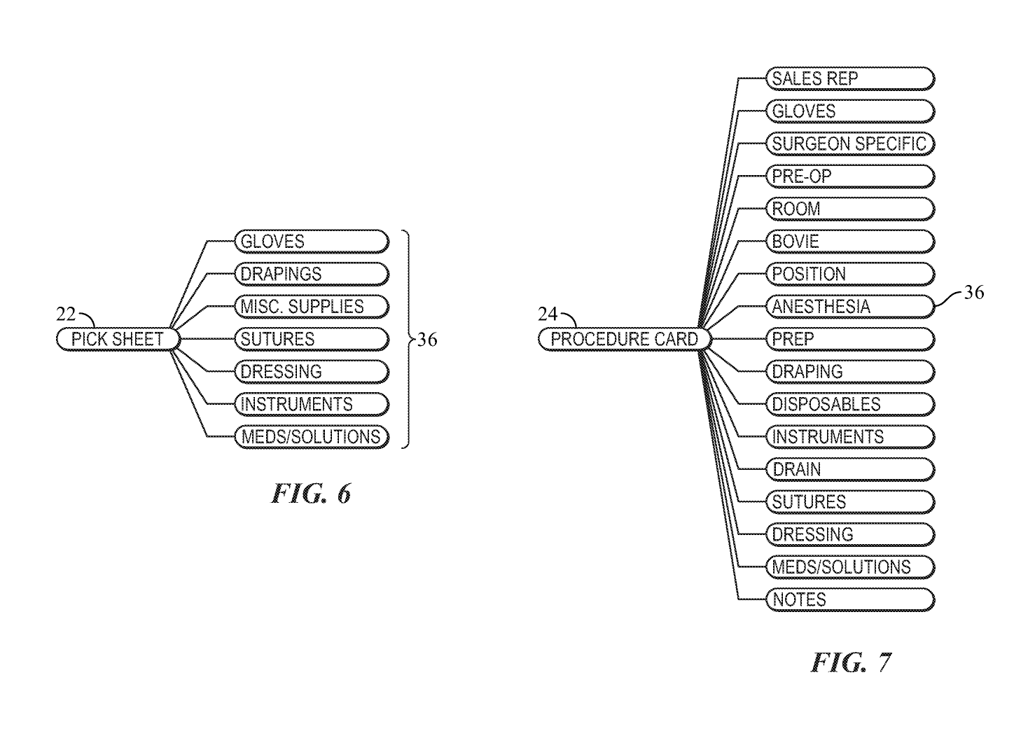

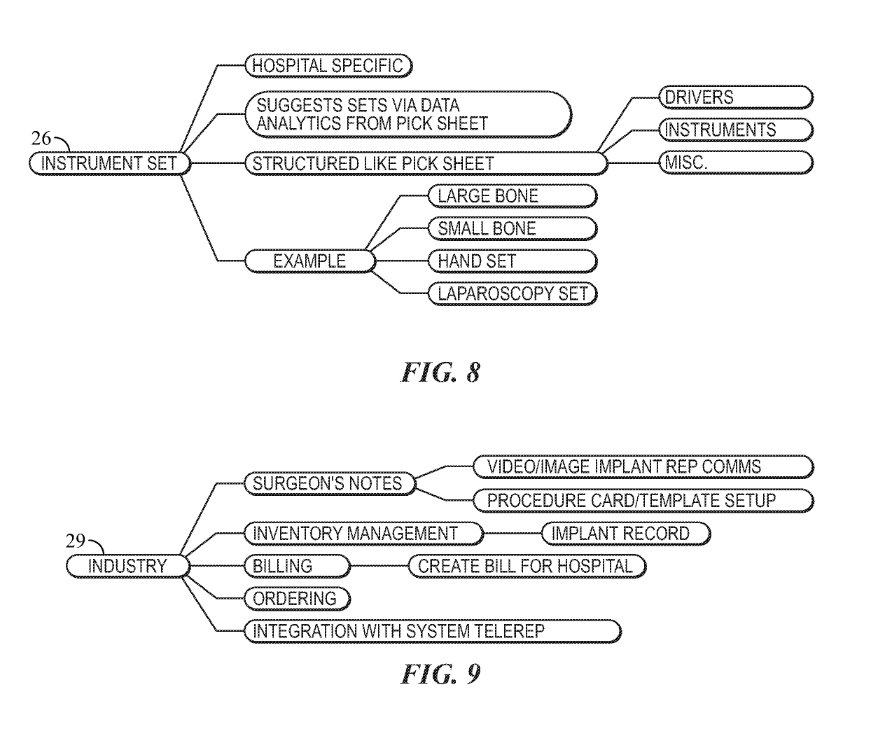

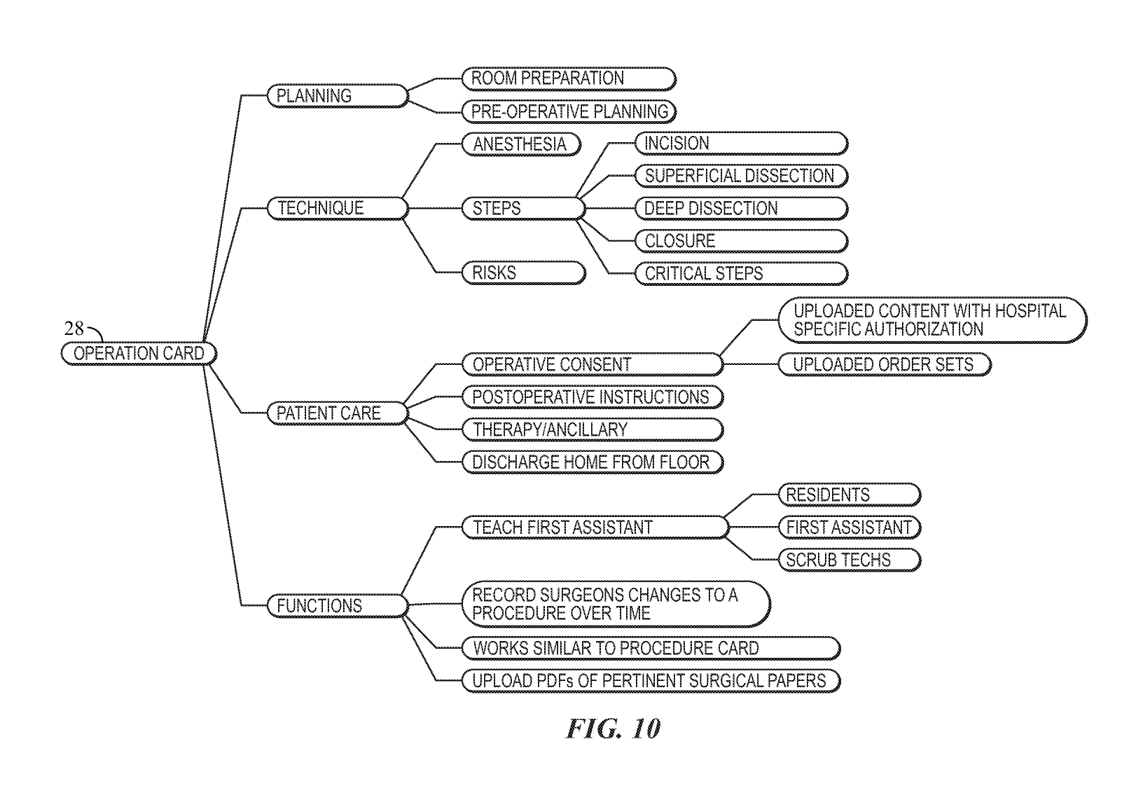

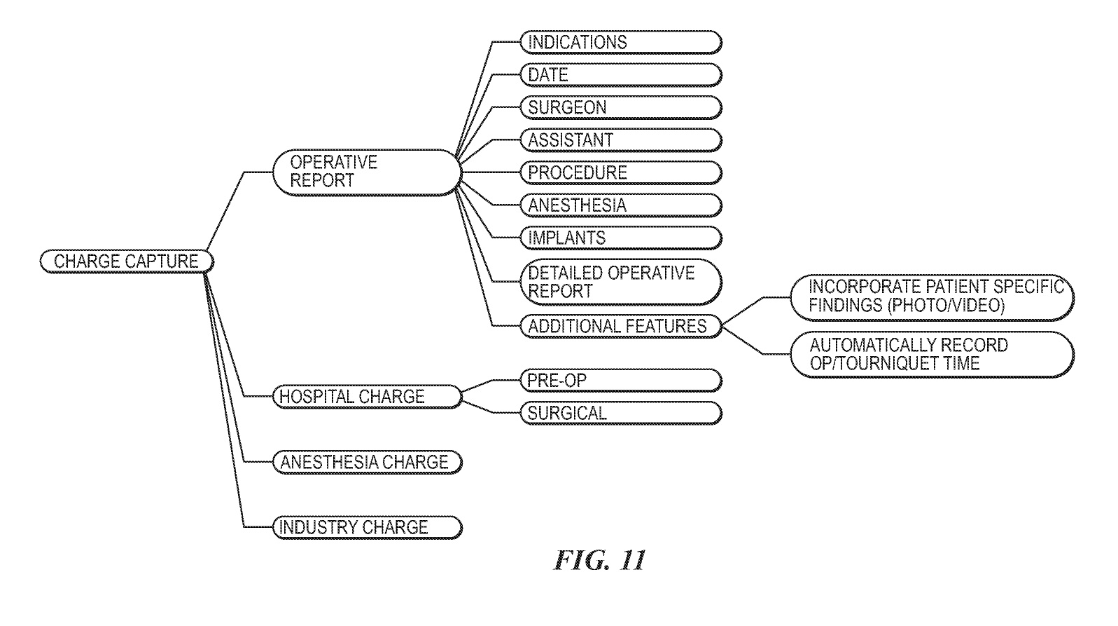

The recommender module 18 includes a collaborative filter 19 to enable user-to-user recommendation of data entry field items 20 in the scalable surgical coordination templates based on past and the same nodes (i.e. inputs). These similarities with the data entry fields are used to pre-populate data entry fields with the pick sheets, procedure cards, instrument set sheets, industry sheets, and operations card (shown in FIGS. 3-5C).

The system 10 can communicate with remote devices (e.g. other computer or smart devices) over a network such as remote desktop interface, Ethernet adapters, and Local Area Network adapters. The system 10 may include a web browser for retrieving web pages or other language streams and present those pages visually or aurally, and/or otherwise to execute scripts controls and other codes on those pages accepting the user inputs with respect to the pages/streams. The webpage or markup language may include HTML or other conventional forms such as XML, scripts, controls or may include a web server for delivering web content directly to the clients or medical centers.

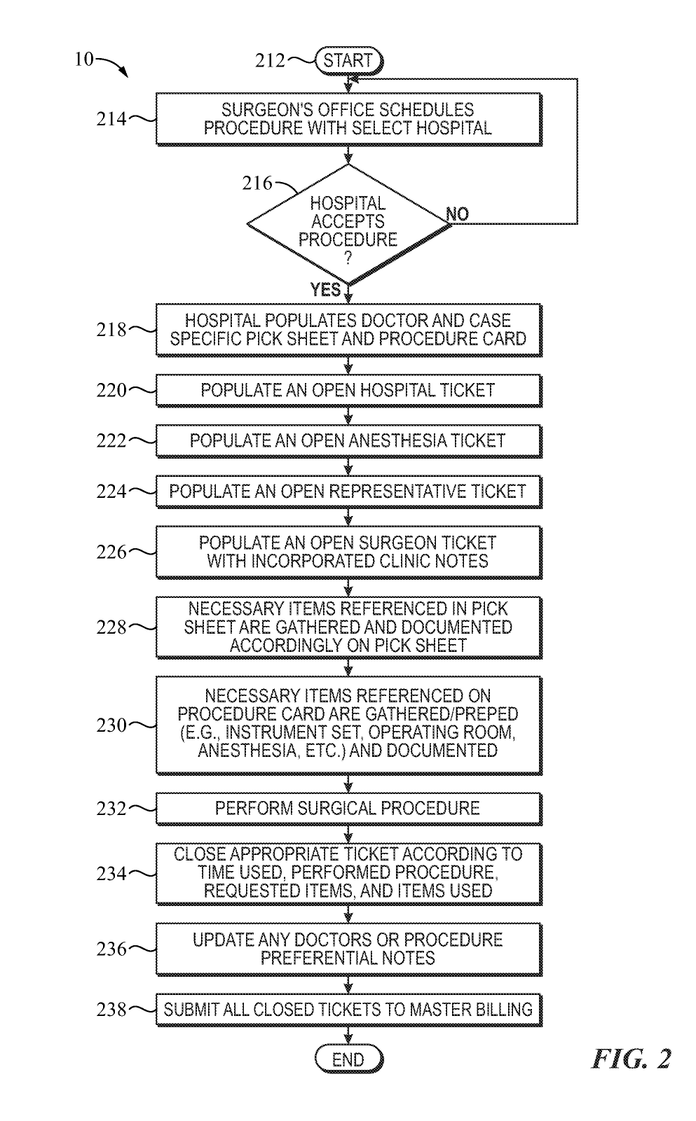

Shown in FIG. 2 is a flow chart illustrating a method of using the system 10 to create a scalable surgical coordination template 20. The system 10 is designed to allow authorized user access through an admistrator to a pluraly of scalable surgical coordination templates whichh include a pick sheet; procedure card, instrument set sheet; industry set sheet; and operation card, and master bill. The system 10 may begin 212 by first obtaining adminstrative permission/access from both the system adminstrator and medical center administrator. Once the proper permissions have been granted, the surgeon or their office staff can log-in and schedule a medical procedure using the scheduling mechanism.

The system's Web-based scheduling mechanism enables the surgeon to schedule a surgery directly with the applicable medical center 214. For example, if Dr. Smith is scheduling a hip replacement surgery (i.e., surgical procedure) at Medical Center A, the system 10 enables Dr. Smith to schedule the surgical procedure, identify the medical device representative, determine the procedure, and specify any special instructions for the procedure such as the use of the live X-ray in the room before the surgery. The scheduling of the surgical procedure automatically opens the surgeon case which remains open until an operative report is generated for the patient and medical center with specific post-operative care instructions. Further, the system 10 will create a master bill which logs the operating room times, medical supplies and any implants used, and notes which interface with the surgeon and other departments and allow the hospital the option to accept/decline the surgery 216. Simultaneously, the system 10 will populate a plurality of surgical templates including the pick sheet, procedure, cards, instrument set sheet, and operations cards 218 to populate an open hospital 220 and generate an anestheia ticket 222 and representative ticket 224. The data field contained within each of these templates enables recommended data input based on the past input and surgeries performed by the surgeon and/or commonly used instruments relating the medical procedure, bone set, etc.

Each of the plurality of surgical cards is accessible by an authorized user who is further defined as a surgeon, surgical tech, resident, anesthesia provider, medical device representative, surgical nurse, scrub tech, or hospital administrative staff who may access, edit and collaborate any edits or modification in each of the plurality of surgical cards 18.

Upon scheduling by the surgeon, a master surgical template is created which enables Dr. Smith to select “total hip replacement” from a drop-down menu of surgical procedures. Further, Dr. Smith can select Representative 1 as the selected medical representative, which provides a responsive calendar invite with the pertinent date, time, and special instructions from the surgeon. The medical representative ticket will remain open until the case is accepted by said medical representative providing confirmation of receipt back to the surgeon's office.

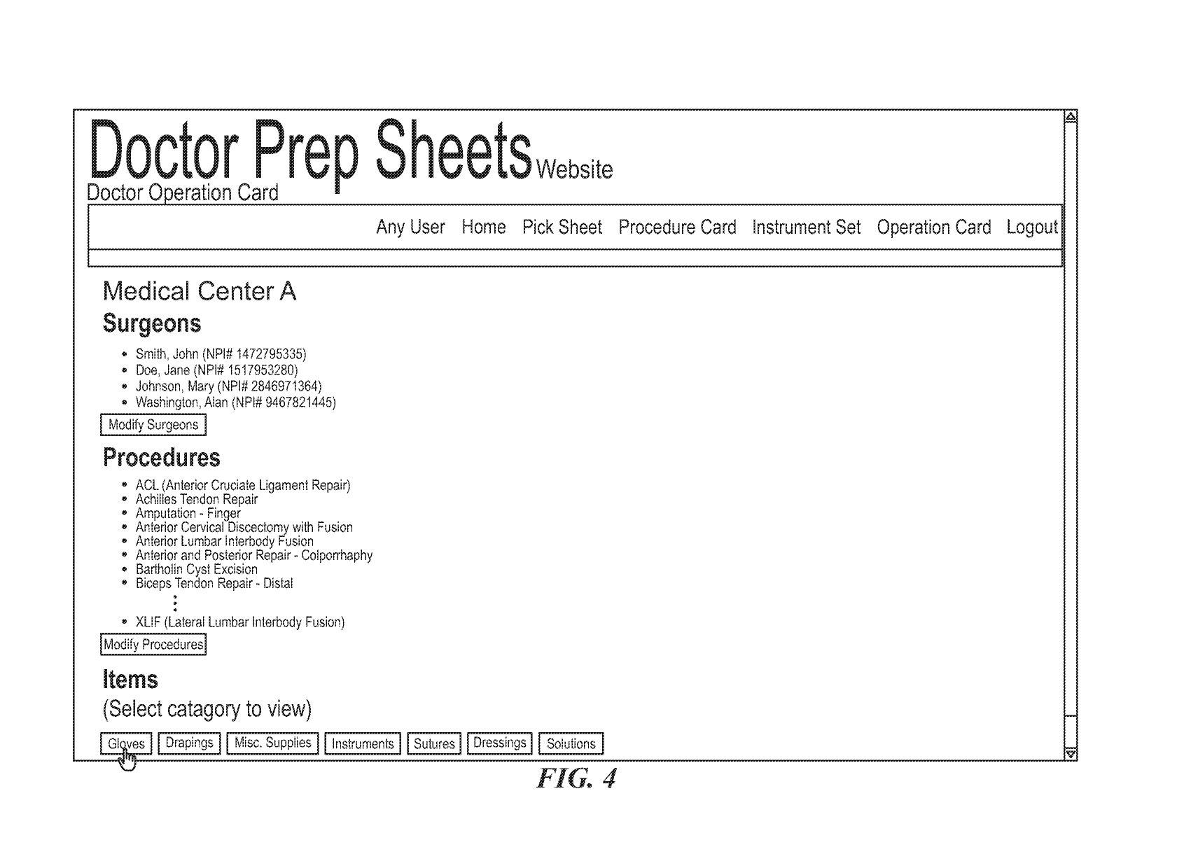

Simultaneously with the scheduling by the surgeon, an editable pick sheet and procedure card (further illustrated in FIG. 3 and FIG. 4) are generated and may be edited by an authorized user. The system 10 further includes a collaborative filter 19 to create a recommended list of instruments based on Dr. Smith's past total hip replacement surgeries. Traditionally, medical centers would provide a generic list of instruments for Dr. Smith to conduct a total hip replacement. The instruments may be a standard number of gloves, drapes, sutures, etc. that are generated from the medical center based on surgery or bone set. The system 10 described includes a recommender module 18 with a collaborative filter 19 which provides a recommended list of instruments based on Dr. Smith's surgical habits and past inputs on a plurality of surgical templates wherein new cards are populated using the past inputs of the surgeon and authorized users who have performed the same or similar medical procedures. The instruments are then “pulled” by the medical center material management department who provide the quantity, cost, and hospital number for each of the items pulled from the master inventory list to the pick sheet.

The pick sheet (further illustrated in FIG. 3) provides a list of instruments and materials that are required for Dr. Smith to perform his total hip replacement surgery. The pick sheet is then further sub-divided into categories including gloves, draping's, instruments, instrument sets (pulled in from the instrument set), sutures, dressings, meds solutions. The instruments provided in the pick sheet can be arranged specific to a facility or hospital to allow the material management staff to efficiently locate and “pick” required instruments.

The system 10 is further configured to enable authorized users and material management staff to select missing inventory as “needed” from a drop-down menu. Once the instrument or material is marked as “needed,” a new category will be generated such that subsequent authorized users or staff will be able to efficiently identify and order the “needed” instruments and materials prior to the time of the scheduled surgical procedure. Traditional “pick sheets” lacked this action item and often left staff having to sort through hundreds of instrument and materials to identify what is missing and requiring action prior to surgery. Further, the system 10 identifies who has “picked” the specific instruments and materials to create a central point of contact which is viewable on a hand-held smart device. As mentioned above, the collaborative filter 19 further creates a recommended list of instruments and materials based on the surgeon and previously generated pick sheets.

At the time of Dr. Smith's scheduling of the total hip replacement, a procedure card is generated (further illustrated in FIG. 4). The procedure card provides a step-by-step instruction of how the surgical procedure should be prepared which is not provided in the “physician orders.” The procedure card is designed for use during the pre-operative stage through each facet of the procedure and utilized by those authorized users who prepare the patient for the surgical procedure, as well as separate authorized users who are present during the surgical procedure and during the recovery time

The system 10 and procedure card enables the authorized user to collaborate amongst one another by providing notes, files, images, and video to describe various preferred strategies, preparation techniques, and best practices based on the procedure and surgeon. For example, when conducting the total hip replacement, a surgical tech may upload and share a video of the preferred way Dr. Smith would like the patients displayed so that the leg is properly shaven and there are not visible cuts or scars shown. The procedure card further contains information such as contact information for medical sales representatives, the preferred placement of instruments and materials in the pre-operation and operation rooms, as well as surgeon and anesthesia specific information and preferred methods of preparation.

As with any surgery or medical procedure and management methods, there is a level of immutable information that carries across all procedures for a specific surgeon. Similar to the above-mentioned scheduling methods, the procedure is a template-oriented surgical card. For example, when Dr. Smith creates a procedure card for the total hip replacement this creates a master template that can be easily modified by Dr. Jones to meet their specific differences and needs when conducting a total hip replacement. Once Dr. Jones has made edits, these edits are added to the master template. The template method enables hospital administration to monitor variations from the templates by surgeons, provides surgeons with the knowledge of peer surgical methods and best practices, and allows for efficiency in the creation and maintenance of such cards and processes. As previously discussed, the permission level which is granted by both the system administration and medical center administration allows both Dr. Smith and Dr.

Jones to log-in to the system and make edits and modifications to already existing procedure cards. Further, Dr. Smith or Dr. Jones may share these edited procedure cards with other surgeons or medical centers.

Similar to Dr. Smith and Dr. Jones, the designated anesthesiologist (for instructional purposes only, Dr. Day) will have a separate log-in and permissions which enable her to “pull” and edit the specific instructions on the procedure card. Further, Dr. Day may be able to view pictures or videos on how to efficiently use a specific instrument or view clinical research documents relating to the medical procedure. In another example, Nurse Johnson will be able to log-in and edit the procedure cards for the Operating Room nurses to ensure all instruments and materials are organized and arranged according to Dr. Smith's preferred manner. Each of these authorized users can collaborate amongst others to provide the most efficient and preferred manner of preparing patients for surgical procedures.

Further generated at the time of scheduling a medical procedure is the hospital-specific instrument sets. The instrument set allows the hospital to create and modify their own existing instrument sets and materials using the collaborative filter 19 which factors in the most commonly used instruments for a similar type procedure within a hospital. For example, if Dr. Smith is to perform a femur repair, the instrument set function will collate instruments from the pick sheet into a set or a box of instruments to simplify the process of preparing for surgery. For instance, if Dr. Jones is performing a femur repair, a set of instruments can be created that group instruments and surgical tools that are commonly used together. The instrument set information is further viewable on a smart device over a network. The second major function of the instrument set function is to utilize data analytics to collate and suggest the creation of new hospital-specific sets based on common instruments used in similar procedures by different surgeons. Accurate and current instrument sets create efficiencies in the sterilization and material management processes.

The industry set is primarily designed for the industry and medical representatives. Every surgeon has a specific manner which they utilize for a specific industry implant system from a specific medical device company. For many cases, there is a requirement for industry or medical representatives to bring their own industry sets with them inside the operating room to help ensure assembly is proper, or provide instruction in the most efficient methods of the using the equipment. The surgical template directed at industry sets ensures that the surgeon's preferred instruments and equipment are present for the surgery. Traditionally, industry and medical representatives keep “surgeon's notes” which provide guidance when preparing for a case with a specific surgeon. These “surgeon notes,” unlike the pick sheet cards, are not specific to the medical center, but rather to the specific surgeon regardless of facility. These industries set sheets of the surgical sheets are designed to create a surgeon-specific procedure card for how each surgeon utilizes the Industry Set and equipment provided. Further, the industry set sheet 29 provides guidance from the industry or medical representative to the scrub tech who is responsible for setting up the instruments, equipment, and materials for the surgical procedure. These collaborative instructions may be brief picture/video clips or text explanations that the scrub tech can utilize to prepare for the case and assemble the necessary instruments. Lastly, the system 10 further enables the surgeon or authorized user in the operating room to conduct a live interface with an industry or medical representative without requiring the representative in the operating room for each medical procedure while receiving real-time feedback on medical device assembly and techniques.

The system 10 further includes master bill and ordering system. This section creates a master bill, drawing from the “Open” section of the pick sheet and the time tracking section of the procedure card. This can generate surgery charges as well as anesthesia charges. From the “OPEN” column of the pick sheet a modifiable “USED” category is created which can be simply up-ticked or down-ticked for charge capture. From a surgical perspective, this is easily modified as new supplies are opened or utilized and if an item is used that is not on the pick sheet, it can easily be added by typing in the first couple of letters of the item which quickly identifies the item and populates the pick sheet since the hospital-specific database exists.

It will be appreciated by persons skilled in the art that the present embodiment is not limited to what has been particularly shown and described hereinabove. In addition, unless mention was made above to the contrary, it should be noted that all the accompanying drawings are not to scale. A variety of modifications and variations are possible considering the above teachings without departing from the following claims.

What is claimed is:

1. A surgical management and coordination computer system, the system comprising:
a microprocessor having a memory module and configured to provide a plurality of scalable surgical coordination templates comprised of at least one user input specific to a particular procedure and selected by an authorized user using a forced function mode to increase surgical management efficiency and standardize a pre-selected surgical procedure.

2. The system of claim 1, wherein the plurality of scalable surgical templates includes:
a pick sheet;
a procedure card;
an instrument set sheet;
an industry set sheet;
an operation card; and
a master bill.

3. The system of claim 1, wherein the plurality of authorized users includes:
a surgeon;
a surgical tech;
a resident;
an anesthesia staff member;
a medical device representative;
a surgical nurse;
a scrub tech; and
a hospital administrative staff member.

4. The system of claim 1, further including a scheduling module configured to:
provide a responsive calendar invite to at least one authorized user and a designated medical center when a new surgical ticket is created;
create a pick sheet having a list of recommended instruments based on a predetermined surgical procedure; and
generate a master bill at the designated medical center to capture at least:
an operating time of the predetermined medical procedure from the pick sheet;
a list of materials and instruments used in a predetermined surgical procedure from the pick sheet; and
an anesthesia time entry.

5. The system of claim 4, further including a recommender module to create at least one recommended data entry field on the pick sheet corresponding to a past input of a designated surgeon assigned to the predetermined surgical procedure.

6. The system of claim 5, further configured to provide a recommended instrument set based on the designated surgeon assigned or the predetermined surgical procedure.

7. The system of claim 1, wherein the plurality of surgical templates is editable on a smart device by the plurality of authorized users.

8. The system of claim 1, wherein the pick sheet is further configured to provide a “need category” from a plurality of inventory items displayed in the at least one data entry field of the pick sheet; the “need category” displaying only a desired “needed item” to the plurality of authorized users.

9. The system of claim 1, further configured to display an uploaded file in the memory module onto the at least one of the plurality of surgical templates.

10. The system of claim 1, wherein the uploaded file within the memory module includes:
an instructional media corresponding to an assembly method for an instrument set;
a tutorial corresponding to a preferred method of preparing a patient for the designated surgeon assigned while the patient is in a pre-operative stage; and
a plurality of research information from the designated surgeon assigned to a future surgeon assigned to the predetermined surgical procedure.

11. The system of claim 1, wherein the operations card is further configured to generate an operative report by the designated surgeon assigned to at least a designated patient and a designated medical center based on a performed surgical procedure inclusive of media specific to the designated patient.

12. A surgical management and coordination computer system stored in a non-transitory computer-readable medium, the system comprising:
a microprocessor having a memory module and collaborative filter to:
generate a plurality of scalable surgical coordination templates viewable on a smart device including:
a pick sheet having at least one editable data entry corresponding to an instrument quantity;
a procedure card allowing for an uploaded and stored file to be viewed by an authorized user prior to a predetermined surgical procedure;
an instrument set sheet having a plurality of recommended data entry field based on a designated surgeon assigned or a pre-populated instrument set used at a designated medical center;
an industry set sheet enabling an instructional video stored within the memory module to be uploaded and viewed with the industry set sheet;
an operation card enabling a research file stored within the memory module to be viewed prior to the predetermined surgical procedure by the designated surgeon assigned;
a master bill generated when a new surgical ticket is created by an authorized user and further including a time entry from at least one of the surgical templates including a time of the predetermined surgical procedure which is transmitted to the designated surgeon assigned and the designated medical center.

13. The system of claim 12, further configured to enable a designated administrator to provide an access level to a plurality of authorized user to access, edit and capture a medical charge one at least one a pertinent portion of at least one of the plurality of scalable surgical coordination templates; the plurality of authorized users including:
a surgeon;
a surgical tech;
a resident surgeon or other first assistant to surgery;
anesthesia personnel
a medical device representative;
a surgical nurse; and
a hospital administrative staff member.

14. The system of claim 12, wherein a scheduling of a new surgical ticket further enables:
a responsive calendar appointment to at least one of authorized user to provide a notice of a scheduled medical procedure;
creating a pick sheet and a procedure card having at least one data entry field editable by:
the surgical nurse; and
the surgical scrub tech;
generating a master bill.

15. The system of claim 12, wherein the pick sheet is configured to create a “need category” from a plurality of inventory items displayed in at least one data entry field and subsequently display only the “need category” when opened by an authorized user.

16. The system of claim 15, further configured to generate a purchase order of an item contained in the need category.

17. The system of claim 12, further configured to enable a voice dictation by the designated surgeon assigned to an operative report in the operations card.

18. The system of claim 12, further including a videotelephony mechanism to enable a live interface between an authorized user and an industry representative during the predetermined medical procedure.

19. The system of claim 12, further configured to provide a step-by-step video tutorial of the predetermined medical procedure by the designated surgeon assigned.

20. A method for preparation to perform a medical operation, the method comprising:
a creation of a surgical template to aid in the easy, complete and rapid creation of new pick sheets, procedure/operation cards after the master template and specific for a surgeon user in a facility;
a memory configured to store data relating to the medical operation;
a plurality of user specific logins including an access level designated by an authorized system administrator;
a processor in communication with the memory and configured to:
generate forms in a plurality of phases, each phase related to a stage of preparation for the medical operation, the generating comprising:
in a second phase, generating a pick sheet having data entry fields populated based on past input and current input, the pick sheet providing a list of supplies and supply location to be used for the medical operation; The ability to communicate deficiencies in the pick sheet to other users to rectify before a surgical procedure begins;
in a third phase, generating a procedure card having data entry fields populated based on past input and at least one of current input and data entered to the pick sheet, the procedure card providing a list of steps for medical personnel other than surgeons to follow from preparing a patient for the medical operation to moving the patient to a recovery room;
in a fourth phase, automatic generating an instrument set through the aggregation of instruments garnered from the second phase based on commonality among instruments utilized by different surgeons across similar procedures within a specific facility having data entry fields populated based on past input and at least one of current input and data entered to the pick sheet, the instrument set sheet providing a list of instruments to be packaged together and used by medical personnel including a surgeon to perform the medical operations;
in a fifth phase, generating an industry set sheet having fields populated at least in part by a representative of a manufacturer of a medical device used in the medical operation, the industry set sheet providing information from the manufacturer concerning at least one of an assembly of a medical device, an implantation procedure for the medical device, and an ordering of a medical implant or device for the medical operation;
in a sixth phase, generating an operation card having fields populated by at least one surgeon or delegate, the operation card providing a list of described steps to perform to accomplish the medical operation displayable from a stored file within the memory; and
in a seventh phase that allows for automatic generating of a master bill including an operating/anesthesia time recorded and list of billable materials used during the medical operation found in the pick sheet and the operations card.
